(19) United States
(12) Patent Application Publication
(10) Pub. No.: US 2023/0060688 A1
(43) Pub. Date: Mar. 2, 2023

(54) COMPOSITIONS AND METHODS FOR SKIN RENEWAL

(71) Applicant: Plexus Worldwide, LLC, Scottsdale, AZ (US)

(72) Inventors: Tianan Jiang, Scottsdale, AZ (US); Cynthia Latham, Scottsdale, AZ (US); Stephen Roman, Chandler, AZ (US); Randa Nabaty, Phoenix, AZ (US); Christin Powell, Corte Madera, CA (US)

(73) Assignee: Plexus Worldwide, LLC, Scottsdale, AZ (US)

(21) Appl. No.: 17/977,311

(22) Filed: Oct. 31, 2022

Related U.S. Application Data
(63) Continuation of application No. 16/977,965, filed on Sep. 3, 2020.
(60) Provisional application No. 62/640,440, filed on Mar. 8, 2018.

(51) Int. Cl. A61K 8/73; A61K 8/368; A61K 8/97; A61Q 19/08

(52) U.S. Cl. CPC A61K 8/73; A61K 8/368; A61K 8/97; A61Q 19/08; A61K 2800/522

(57) ABSTRACT

Various embodiments provide topical compositions and methods for treating the skin to modulate the skin microbiome. The application of various embodiments of the topical composition may modulate the growth of various commensal bacterial in the skin microbiome when applied to the skin, which may improve the immune function of the skin and/or reduce premature aging of the skin.

### CROSS REFERENCES TO RELATED APPLICATIONS

This application in a continuation of U.S. patent application Ser. No. 16/977,965, filed Sep. 3, 2020, entitled “Compositions and Methods for Skin Renewal”, which is the National Stage Entry of PCT/US2019/021468, filed Mar. 8, 2019, entitled “Compositions and Methods for Skin Renewal”, which claims priority to, and the benefit of U.S. Provisional Application No. 62/640,440, filed Mar. 8, 2018, entitled “Compositions and Methods for Skin Renewal”, all of which are incorporated by reference.

### BACKGROUND

The skin is the human body's largest organ and physical barrier to the environment. The cutaneous surface of the skin is colonized by an ecologically diverse population of microorganisms referred to as the microbiome. The microbial communities colonizing the cutaneous surface are linked to human health and disease.

The skin microbiome may generally comprise resident microbes that persist on the cutaneous surface, re-colonizing after the cutaneous surface is disturbed, and transient microbes that do not establish themselves permanently on the cutaneous surface. The resident and transient microbes are not generally pathogenic in skin of an individual maintaining proper hygiene having an intact skin barrier with normal immune function.

Disease may develop where the physical barrier of the skin is compromised, the individual becomes immune compromised, and/or the microbiota is disturbed. Under such conditions, otherwise commensal microbes on the cutaneous surface may become opportunistic pathogens.

### SUMMARY

Various embodiments provide topical compositions and methods for treating the skin to modulate the skin microbiome. The application of various embodiments of the topical composition may modulate the growth of various commensal bacterial in the skin microbiome when applied to the skin, which may improve the immune function of the skin and/or reduce premature aging of the skin.

### BRIEF DESCRIPTION OF THE DRAWINGS

A more complete understanding of the present technology may be derived by referring to the detailed description when considered in connection with the following illustrative figures. In the following figures, like reference numbers refer to similar elements and steps throughout the figures.

Elements and steps in the figures are illustrated for simplicity and clarity and have not necessarily been rendered according to any particular sequence or scale. For example, steps that may be performed concurrently or in different order are illustrated in the figures help to improve understanding of embodiments of the present technology.

The figures described are for illustration purposes only and are not intended to limit the scope of the present disclosure in any way. Various aspects of the present technology may be more fully understood from the detailed description and the accompanying drawing figures, wherein:

FIG. 1 is a graph illustrating the growth curve kinetics of 10³ CFU/mL of an exemplary strain of S. epidermidis with varying concentrations of Tryptic Soy Broth;

FIG. 2 is a graph illustrating the growth curve kinetics of 10⁵ CFU/mL of an exemplary strain of S. epidermidis with varying concentrations of Tryptic Soy Broth;

FIG. 3 is a graph illustrating the growth curve kinetics of an exemplary strain of S. epidermidis with varying concentrations of FOS;

FIG. 4 is a graph illustrating the growth curve kinetics of an exemplary strain of S. epidermidis with varying concentrations of protocatechuic acid;

FIG. 5 is a graph illustrating the growth curve kinetics of an exemplary strain of S. epidermidis with varying concentrations of cranberry extract;

FIG. 6 is a graph illustrating the growth curve kinetics based on optical density of an exemplary strain of S. epidermidis with various embodiments of a topical composition; and

FIG. 7 is a graph illustrating the growth curve kinetics based on average Log CFU/mL of an exemplary strain of S. epidermidis with various embodiments of a topical composition.

### DETAILED DESCRIPTION OF EXEMPLARY EMBODIMENTS

The present technology may be described herein in terms of functional block components and various processing steps. Such functional blocks may be realized by any number of components configured to perform the specified functions and achieve the various results. For example, the present technology may employ various types of topical compositions for use on the skin. In addition, the present technology may be practiced in conjunction with any method for formulating topical compositions and/or any system or device for applying topical compositions to the skin and the system described is merely one exemplary application for the technology.

Formulations and methods for producing a topical composition according to various aspects of the present technology may comprise a skin microbiome modulating composition. The skin microbiome modulating composition may be a topical composition for application to a skin cutaneous surface that may modify the growth of commensal bacteria of the skin microbiome. In various embodiments, the skin microbiome modulating composition may comprise bioactive ingredients that may maintain and/or stimulate the growth of beneficial bacteria in the skin microbiome. For example, in some embodiments, the skin microbiome modulating composition may promote the growth of the commensal bacteria Staphylococcus epidermidis (S. epidermidis).

Colonization of S. epidermidis in the skin microbiome may affect the immune function of the skin and/or reduce premature aging of the skin. S. epidermidis promotes restoration of healthy skin, in part, by preventing colonization of the skin microbiome with pathogenic microbes. S. epidermidis has also been shown to influence host immunity by boosting the host immunity against S. aureus, activating mast cell-mediated immunity, suppressing uncontrolled inflammatory reactions during wound healing, inducing skin's adensosine monophosphate (AMP) production, and stimulating cutaneous T-cell maturation. Accordingly, S. epidermidis may work in cooperation with the host defense system and endogenous AMPs to protect the skin. Moreover, the microbiome may represent a kind of filter for the environment as most agents in contact with and/or penetrating through the skin are also in contact with the microbiota.

Various embodiments of the skin microbiome modulating composition applied to the skin may increase the colonization of S. epidermidis on the skin, resulting in protecting the skin from premature aging and reducing signs of aging such as reducing hyperpigmentation, wrinkles, and inflammation and improving the adaptive capability of the skin. The skin microbiome modulating composition may also protect the skin from oxidative damage from the environment. Oxidative damage from ultraviolet irradiation from the sun and air pollution may cause deleterious effects in human skin, including sunburn, immune suppression, and premature aging such as photoaging which may be characterized in part by wrinkles, altered pigmentation, and loss of skin tone.

Various embodiments of the topical composition may comprise a suitable combination of plant polyphenols, antioxidants, phenolic acids and/or metabolites, and/or prebiotics. In an exemplary embodiment, the skin microbiome modulating composition may comprise oligofructose, a cranberry extract, and/or a phenolic acid. In some embodiments, a variety of additives may be mixed with the skin microbiome modulating composition. Additives may be added to provide any desired physical property to the topical composition comprising the skin microbiome modulating composition, such as modifying viscosity, pH, spreadability, water resistance. For example, additives may comprise any suitable moisturizer, sunscreen, emollient, emulsifier, colorant, fragrance.

Various exemplary embodiments of the skin microbiome modulating composition may comprise a prebiotic. The prebiotic may comprise any prebiotic or combination of prebiotics suitable for topical application to the skin that support the growth of S. epidermidis. In various embodiments, the prebiotic may comprise fructooligosaccharides (FOS), oligofructose, oligosaccharides, galacto-oligosaccharides (GOS), inulin, xylo-oligosacchrides (XOS), arabinoxylan-oligosacchrides (AXOS), isomalto-oligosacchrides (IMOS), gluco-oligosaccharides, soy-oligosaccharides, pyrodextrin, lactosucrose, polydextrose, lactulose, raffinos, lactitiol, xylan, gentiobiose, and/or pullulan.

In some embodiments, the prebiotic of the skin microbiome modulating composition may comprise an oligofructose. Oligofructose (also called Fructo-oligosaccharide or FOS) is a carbohydrate consisting of oligosaccharides composed of fructose units linked together by β-(2,1)-linkages. Part of the fructose chains are terminated by a glucose unit. For example, the degree of polymerization may range between two and eight fructose and glucose units. In an exemplary embodiment, FOS may be produced by the partial enzymatic hydrolysis of chicory inulin. In addition to increasing the growth of S. epidermidis, FOS may also act as a humectant and/or moisturizer in the skin.

In a screening study, the effect of xylo-oligosaccharides (XOS) xylan, galacto-oligosaccharide (GOS), fructo-oligosaccharide (FOS), polydextrose (PDX), lactitol, gentiobiose, and pullulan on the growth of S. epidermidis was investigated in pure cultures in vitro. FOS was only prebiotic in the screen to stimulate the growth of S. epidermidis, demonstrating that not all prebiotics promote growth of S. epidermidis.

Various exemplary embodiments of the skin microbiome modulating composition may comprise polyphenols such as proanthocyanidins (also known as procyanidins). For example, the proanthocyanidins may comprise catechin, epicatechin, procyanidin A1, procyanidin 2, procyanidin B, procyanidin Cl, procyanidin 2, proanthocyanidin type-A, proanthocyanidin type-B, oligomeric proanthocyanidins (OPC), and/or other oligomeric flavonoids. Various embodiments of the proanthocyanidins may be found in plants such as maritime pine bark and most other pine species, cinnamon, cranberry, peanut skins, apple, blueberry, aronia, cocoa beans, grape seed, grape skin (procyanidins and prodelphinidins) and red wines, bilberry, black currant, hawthorn, rosehip, sea buckthorn, green tea, black tea, and other plants and fruits.

In some embodiments, the proanthocyanidins in the skin microbiome modulating composition may comprise a cranberry extract. The cranberry extract may comprise an extract of cranberry fruit using any suitable solvent such as water. In some embodiments, the cranberry extract may further comprise any suitable acidity regulator, such as magnesium hydroxide for absorbing moisture, and/or any suitable anticaking agent, such as a tricalcium phosphate carrier. In one embodiment, the cranberry extract may be approximately 15% by weight proanthocyanidins.

Various exemplary embodiments of the skin microbiome modulating composition may comprise a phenolic acid. The phenolic acid may exhibit antibacterial, anti-inflammatory, anti-viral, and/or anti-aging activity. In various embodiments, the phenolic acid may comprise protocatechuic acid (PCA), 3,4-dihydroxy benzoic acid, p-hydroxybenzoic acid, gallic acid, vanillic acid, syringic acid, o-coumaric acid, m-coumaric acid, p-coumaric acid, caffeic acid, ferulic acid, sinapinic acid, chlorogenic acid, and/or ellagic acid.

In some embodiments, the phenolic acid may comprise PCA. PCA is 3,4-dihydroxy-benzoic acid found in many edible and medicinal plants. In one embodiment, PCA and Cranberry extract may exhibit anti-biofilm activity against S. aureus but not S. epidermidis.

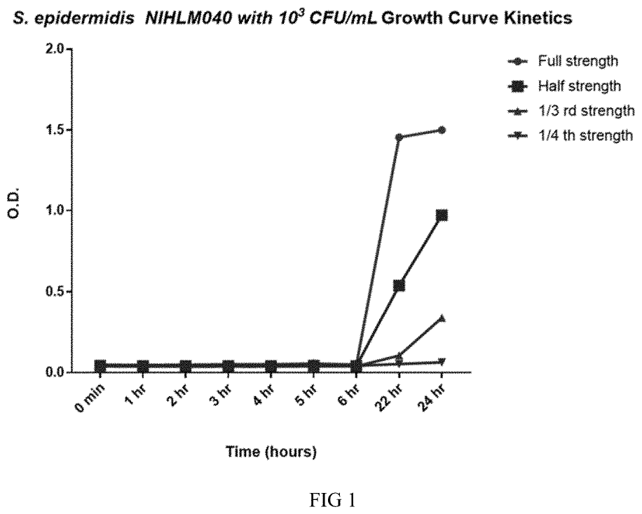

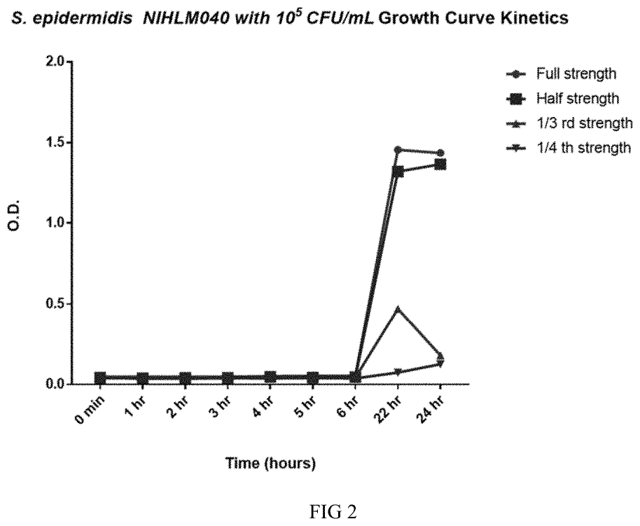

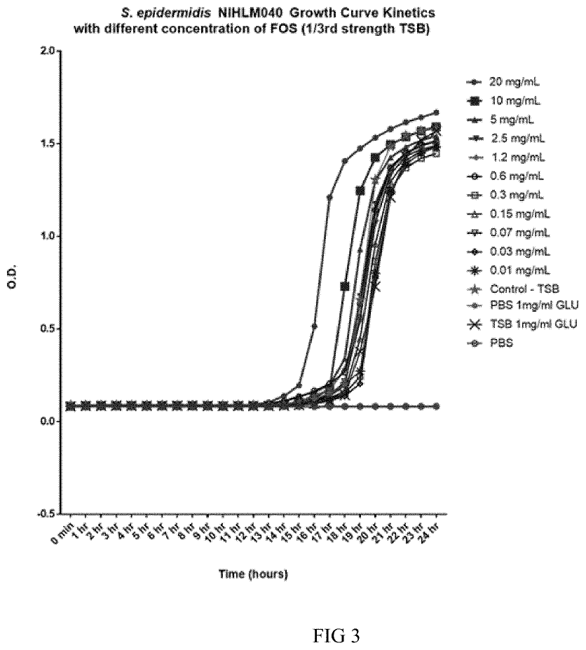

The effect of various embodiments of the skin microbiome modulating composition were studied on a strain of S. epidermidis. Referring to FIGS. 1 and 2, a strain of S. epidermidis was isolated from the facial skin of a human subject. The effect of different concentrations of Tryptic Soy Broth growth medium (TSB medium) were evaluated to optimize growth conditions of the isolated strain of S. epidermidis. FIG. 1 illustrates the growth curve kinetics of a starting inoculum of 1×10³ colony forming units per milliliter (CFU/mL) of S. epidermidis in TSB medium. FIG. 2 illustrates the growth curve kinetics of a starting inoculum of 1×10⁵ CFU/mL of S. epidermidis in TSB medium. In both starting inoculum concentrations, one third strength of TSB medium minimally supported the growth of S. epidermidis. One third strength of TBS medium was accordingly used in the studies shown in FIGS. 3-7.

Referring to FIG. 3-5, the effect of cranberry extract, FOS, and PCA were evaluated separately for their effect on the growth of S. epidermidis suspension cultures. The optical density of the S. epidermidis cultures were measured each hour for 24 hours. Specifically, the effects of the following ingredients were evaluated:
1. Cranberry extract: CE15 Cranberry PE 15% proanthocyanidins BL-DMAC, Naturex
2. FOS: Orafti® P95, Oligofructose, Beneo
3. PCA: Protocatechuic acid, Cayman Chemicals

Referring to FIG. 3, the concentrations of FOS in milligram per milliliter (mg/mL) of one third strength TSB medium that were evaluated for growth of were S. epidermidis were: 20, 10, 5, 2.5, 1.2, 0.6, 0.3, 0.15, 0.07, 0.03 and 0.01 mg/mL. Concentrations of 10-20 mg/mL FOS resulted in significantly higher growth of S. epidermidis.

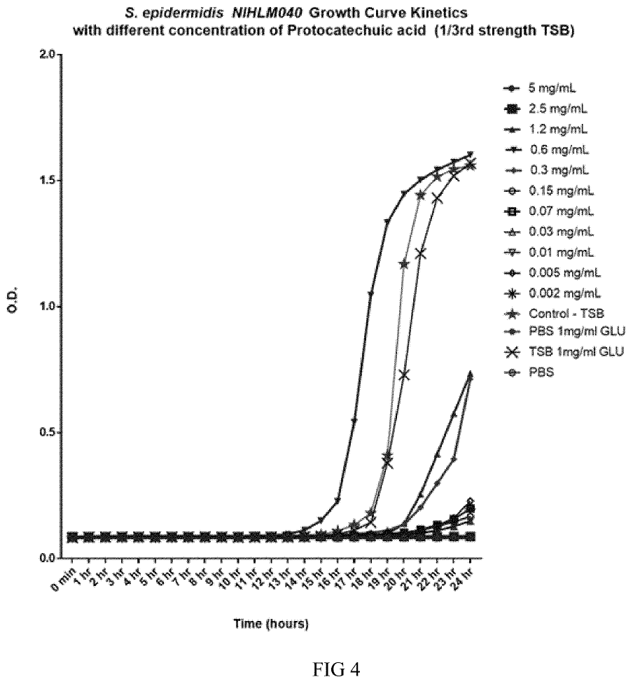

Referring to FIG. 4, the concentrations of PCA in mg/mL of one third strength TSB medium that were evaluated for growth of were S. epidermidis were: 5, 2.5, 1.2, 0.6, 0.3, 0.15, 0.07, 0.03, 0.01, 0.005 and 0.002 mg/mL. PCA of 0.6 mg/mL showed similar growth of S. epidermidis to the control TBS medium.

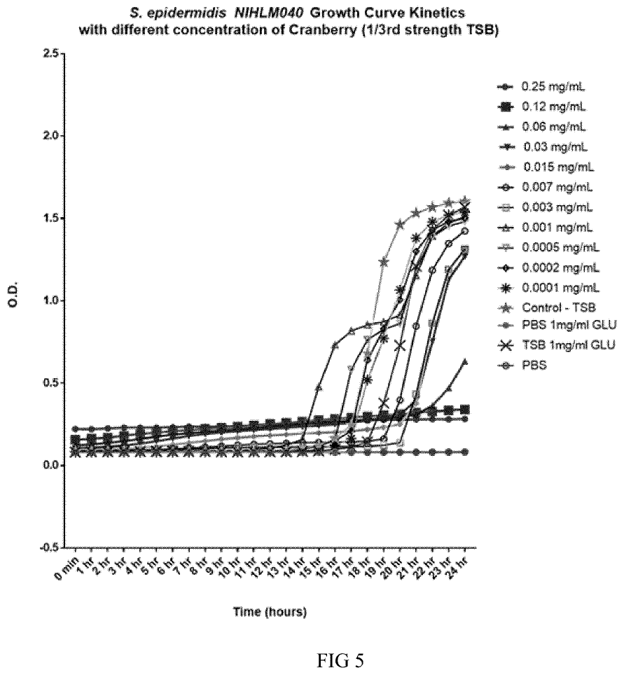

Referring to FIG. 5, the concentrations of cranberry extract in mg/mL of one third strength TSB medium that were evaluated for growth of were S. epidermidis were: 0.25, 0.12, 0.06, 0.015, 0.007, 0.003, 0.001, 0.0005, 0.0002 and 0.0001 mg/mL. In comparison with control TSB medium, there was dose dependent inhibition of S. epidermidis growth in the presence of the cranberry extract.

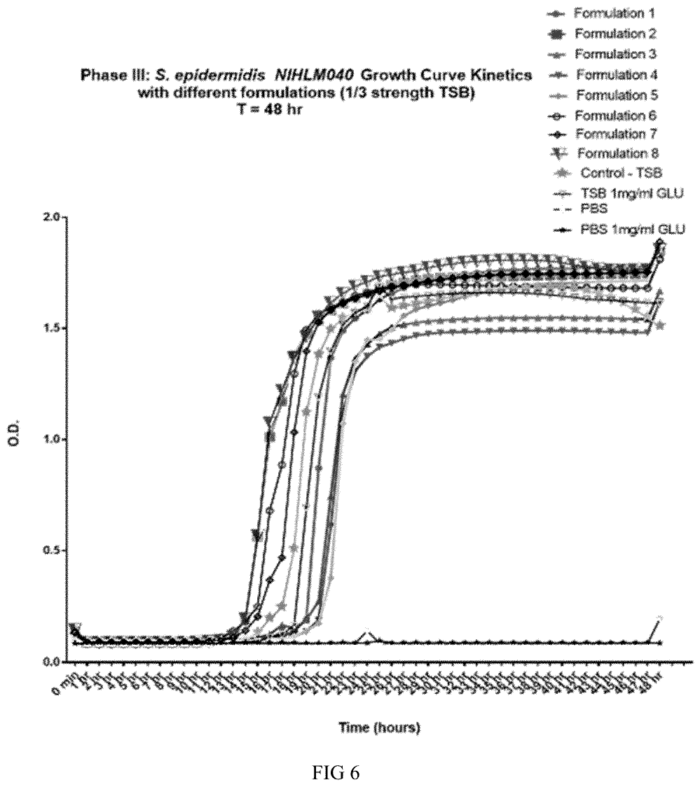

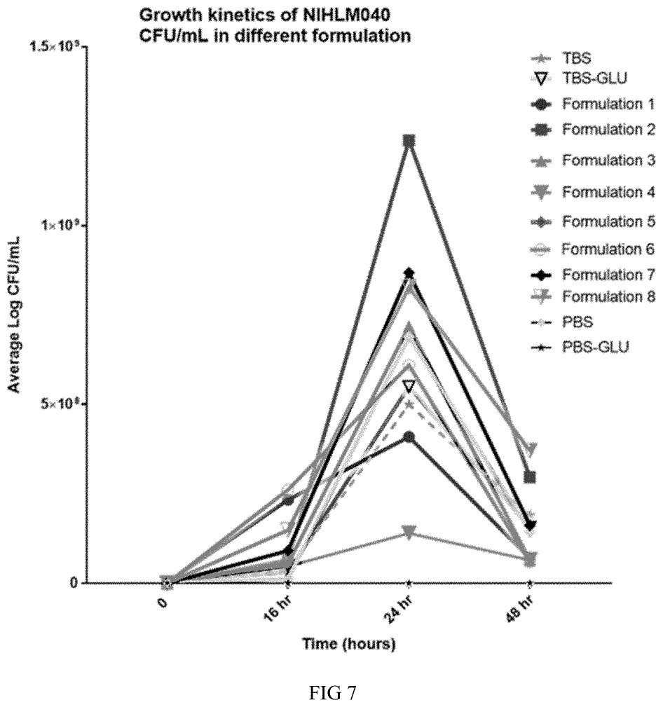

Referring to FIGS. 6 and 7, various embodiments of the skin microbiome modulating composition comprising various concentrations of PCA, FOS, and cranberry extract were evaluated. The various embodiments of the skin microbiome modulating composition are represented in Table 1 as formulations 1 through 8:

TABLE 1

| | | | | CFU/mL | % Growth increase at |
|---|---|---|---|---|---|
| | PCA | Cranberry | FOS | Log 10 at | 24 hours when compared |
| Formulation No. | (mg/mL) | Extract | (mg/mL) | 24 Hr | to control TSB medium |
| 1 | 0.3 | 0.0001 | 10 | 410000000 | No significant increase |

EXAMPLE FORMULATIONS

| | | | | | |
|---|---|---|---|---|---|
| 2 | 0.6 | 0.0001 | 10 | 1240000000 | 148% |
| 3 | 0.3 | 0.0001 | 25 | 720000000 | 44% |
| 4 | 0.3 | 0 | 25 | 140000000 | No significant increase |
| 5 | 0.3 | 0 | 0 | 690000000 | 38% |
| [symbol] 6 | 0 | 0 | 25 | 610000000 | 22% |
| 7 | 0 | 0 | 10 | 870000000 | 74% |
| 8 | 0.6 | 0 | 0 | 830000000 | 66% |
| 9 | | | | 500000000 | |
| [symbol] 10 | | | | 550000000 | |

[symbol]

indicates data missing or illegible when filed

Referring to FIG. 6, based on optical density of the cultures at 16 hours after inoculation, formulations 2, 6, 7, and 8 significantly increased the growth of S. epidermidis by 86%, 87%, 84%, 89% respectively, in comparison with the control TSB medium. The change in CFU/mL of S. epidermidis growth in the presence of each of formulation 1 through 8 was measured after 24 hours incubation, as shown in Table 1. In comparison with the control TBS medium, the increase in CFU/mL was highest with formulation 2 with 148% increase in growth after 24 hour of incubation. In comparison with the control TBS medium, the percentage growth increase at 24 hours with formulations 6, 7, and 8 was 22%, 74%, and 66%, respectively.

TABLE 2

| Formulation No. | CFU/mL | LOG10 |
|---|---|---|
| 1 | 4.1 × 10⁸ | 8.6 |
| 2 | 1.24 × 10⁹ | 9.1 |
| 3 | 7.2 × 10⁸ | 8.9 |
| 4 | 1.4 × 10⁸ | 8.1 |
| 5 | 6.9 × 10⁸ | 8.8 |
| 6 | 6.1 × 10⁸ | 8.8 |
| 7 | 8.7 × 10⁸ | 8.9 |
| 8 | 8.3 × 10⁸ | 8.3 |
| PBS | 0 | 0.0 |
| PBS + Glucose | 0 | 0.0 |
| TSB | 5.0 × 10⁸ | 8.7 |
| TSB + Glucose | 5.0 × 10⁹ | 8.7 |

As supported by the results of FIGS. 6 and 7 and Table 2, one embodiment of the skin microbiome modulating composition that may promote in vitro growth of the beneficial microbe S. epidermidis may comprise approximately 0.0001 mg/mL cranberry extract, 10 mg/mL FOS, and 0.6 mg/mL PCA. This embodiment showed an increase of in vitro growth of S. epidermidis in CFU/mL by 177%, 148%, and 55.5% at 16, 24, and 48 hours after treatment, respectively when compared to the TSB control. Additionally, various embodiments of the skin microbiome modulating composition may comprise approximately 0.3-0.6 mg/mL PCA, 0.0001 mg/mL cranberry extract, and 10-25 mg/mL FOS.

Various embodiments of the topical composition comprising the skin microbiome modulating composition may comprise various additives. For example, additives may comprise fragrance, antioxidants, emollients, sunscreens, skin lighteners, pore reducers, exfoliators, acids for reducing hyperpigmentation, and/or emulsifiers to reduce tackiness. In one embodiment, the topical composition may comprise the skin microbiome modulating composition and further comprise an ingredient such as β-glucan for inhibiting the growth of pathogenic bacteria including Staphylococcus aureus.

In some embodiments, the topical composition may comprise the skin microbiome modulating composition along with various additives that promote firming of the skin. Some embodiments of the skin firming composition may comprise the skin microbiome modulating composition and additives such as argerline, ceramides for moisture, hyaluronic acid, niacinamide B3 for lightening skin, and/or red clover flower extract for reducing pore size.

In another embodiment, the topical composition may comprise the skin microbiome modulating composition along with various additives that promote exfoliation of the skin. Some embodiments of the skin exfoliating composition may comprise the skin microbiome modulating composition and additives such as retinol, azalic acid to reduce hyperpigmentation, lactic acid, Matrixyl, and/or Nigerian hibiscus.

Suitable antioxidants may include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-carotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., Ionox 100), 2,4-(tris-3′,5′-bi-tert-butyl-4′-hydroxybenzyl)-mesitylene (i.e., Ionox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivatives, vitamin Q10, wheat germ oil, zeaxanthin, or combinations thereof. One skilled in the art will appreciate that the antioxidants incorporated into the composition (including those listed herein) encompass all potential salt and ester forms of the antioxidants in addition to the pure forms of the compound. In some embodiments, the antioxidant may comprise a vitamin E compound such as tocopherol acetate, tocopherol linoleate, tocopherol nicotinate, tocopherol succinate, ascorbyl tocopherol phosphate, dioleyl tocopherol methylsilanol, tocophersolan, and tocopherol linoleate/oleate. In one embodiment, included in the vitamin E oil are traces of safflower oil, and other oils. In another embodiment, the vitamin E formula further comprises the largest amount of sunflower seed oil followed by safflower seed oil, tocopheryl acetate, rice bran oil, almond oil, apricot oil, wheat germ oil and lecithin.

Various exemplary embodiments of a topical composition comprising the skin microbiome modulating composition may comprise water, Glycerin, Glyceryl Stearate SE, Dimethicone, Isosorbide Dicaprylate, Squalane, Niacinamide, Magnolia Glauca Flower Water, Isopentyldiol, Acetyl Hexapeptide-8, Hyaluronic Acid, Silanetriol, Dimethiconol, Trifolium Pratense (Clover) Flower Extract, Tetrahexyldecyl Ascorbate, Behenyl Alcohol, Lysolecithin, Sclerotium Gum, Xanthan Gum, Pullulan, Silica, Ethoxydiglycol, Butylene Glycol, Acetyl Tetrapeptide-5, Phenoxyethanol, Ethylhexylglycerin, Polylactic Acid, Cichorium Intybus (Chicory) Root Oligosaccharides, Bismuth Oxychloride, Ethylhexyl Hydroxystearate, Panthenol, Glycosphingolipids, Glycolipids, Sodium Hyaluronate, Ricinus Communis (Castor) Seed Oil, Hydrogenated Castor Oil, Tocopheryl Acetate, Citric Acid, Caprylic/Capric Triglyceride, Anthemis Nobilis Flower Oil, Jasminum Officinale (Jasmine) Oil, Lavandula Angustifolia (Lavender) Oil, Origanum Majorana Leaf Oil, Santalum Album (Sandalwood) Oil, Vanilla Planifolia Fruit Extract, Cananga Odorata Flower Oil, Bisabolol, Sodium Phytate, Rubus Fruticosus (Blackberry) Seed Oil, 3,4-Dihydroxybenzoic Acid, Magnesium Carboxymethyl Beta-Glucan, Haematococcus Pluvialis Extract, and Vaccinium Macrocarpon (Cranberry) Fruit Extract.

Various exemplary embodiments of a topical composition comprising the skin microbiome modulating composition may comprise Potassium Azeloyl Diglycinate, Dicaprylyl Ether, Caprylic/Capric Triglyceride, Glycerin, Isosorbide Dicaprylate, Glyceryl Monostearate, Butyrospermum Parkii (Shea Butter), Phytic Acid, Rosa Damascena Flower Water, Cetyl Alcohol, Glyceryl Distearate, Steareth 21, Dimethyl Isosorbide, Hydroxypinacolone Retinoate, Vaccinium Macrocarpon (Cranberry) Fruit Extract, Cichorium Intybus (Chicory) Root Oligosaccharides, Lysolecithin, Sclerotium Gum, Xanthan Gum, Pullulan, Silica, Hydroxypropyl Cyclodextrin, Palmitoyl Tripeptide-38, Lactic Acid, Ethoxydiglycol, Squalane, Phenoxyethanol, Ethylhexylglycerin, Dimethicone, Bismuth Oxychloride, Ethylhexyl Hydroxystearate, Bakuchiol, Hibiscus Sabdariffa Flower Extract, Sodium Hyaluronate, Ricinus Communis (Castor) Seed Oil, Hydrogenated Castor Oil, Sodium Hydoxide, Rosa Rubiginosa Seed Oil, Rosa Damascena Flower Oil, Allantoin, Tocotrienols (Tocomin), Tocopherol, Elaeis Guineensis (Palm) Oil, Sodium Phytate, Brassica Oleracea Italica (Broccoli) Seed Oil, Myrciaria Dubia FruitExtract, 3,4-Dihydroxybenzoic Acid, and Magnesium Carboxymethyl Beta-Glucan.

Various embodiments of the skin microbiome modulating composition or the topical composition comprising the skin microbiome modulating composition may be implemented into any suitable topical delivery medium formulated for delivery to the skin. In some embodiments, the topical delivery medium may facilitate transdermal delivery of the topical composition. In various embodiments, the topical delivery medium may comprise one or more of a gel, scrub, soap, oil, serum, sunscreen, ointment, cream, lotion, solution, spray, foam, tonic, conditioner, shampoo, and the like.

Various embodiments of the skin microbiome modulating composition may be implemented in a method of modulating the growth of at least one commensal bacterium on the skin of an animal, the method comprising applying an effective amount of a topical composition to the skin of the animal, wherein the topical composition comprises: a prebiotic; a polyphenol; and a phenolic acid; wherein the topical composition is implemented in the topical delivery medium formulated to cause transdermal delivery of the topical composition to the skin of the animal.

In the foregoing description, the technology has been described with reference to specific exemplary embodiments. Various modifications and changes may be made, however, without departing from the scope of the present technology as set forth. The description is to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any appropriate order and are not limited to the explicit order presented in the specific examples. Additionally, the components and/or elements recited in any system embodiment may be combined in a variety of permutations to produce substantially the same result as the present technology and are accordingly not limited to the specific configuration recited in the specific examples.

Benefits, other advantages and solutions to problems have been described above with regard to particular embodiments. Any benefit, advantage, solution to problems or any element that may cause any particular benefit, advantage or solution to occur or to become more pronounced, however, is not to be construed as a critical, required or essential feature or component.

The terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same.

The present technology has been described above with reference to an exemplary embodiment. However, changes and modifications may be made to the exemplary embodiment without departing from the scope of the present technology. These and other changes or modifications are intended to be included within the scope of the present technology.

What is claimed is:

1. A topical composition for application to skin of an animal, wherein the skin comprises at least one commensal bacterium, the topical composition comprising:
a prebiotic comprising a fructooligosaccharide;
a polyphenol; and
a phenolic acid,
wherein the topical composition stimulates in vitro growth of the at least one commensal bacterium thereby increasing an amount of the at least one commensal bacterium on the skin by at least 50% after approximately twenty-four hours of an application of the topical composition to the skin.

2. The topical composition of claim 1, at least one commensal bacterium is Staphylococcus epidermidis.

3. The topical composition of claim 1, wherein the prebiotic comprises at least one of an oligosaccharide, galacto-oligosaccharides (GOS), inulin, xylo-oligosacchrides (XOS), arabinoxylan-oligosacchrides (AXOS), isomalto-oligosacchrides (IMOS), gluco-oligosaccharides, soy-oligosaccharides, pyrodextrin, lactosucrose, polydextrose, lactulose, raffinos, lactitiol, xylan, gentiobiose, and pullulan.

4. The topical composition of claim 1, wherein the polyphenol is at least one proanthocyanidin.

5. The topical composition of claim 4, further comprising a cranberry extract, wherein the cranberry extract comprises the at least one proanthocyanidin.

6. The topical composition of claim 5, wherein the cranberry extract comprises approximately 15% by weight of the at least one proanthocyanidin.

7. The topical composition of claim 4, wherein the proanthocyanidin comprises at least one of catechin, epicatechin, procyanidin A1, procyanidin 2, procyanidin B, procyanidin Cl, procyanidin 2, proanthocyanidin type-A, proanthocyanidin type-B, oligomeric proanthocyanidins (OPC), and oligomeric flavonoids.

8. The topical composition of claim 1, wherein the phenolic acid comprises protocatechuic acid.

9. The topical composition of claim 1, wherein the phenolic acid comprises at least one of 3,4-dihydroxy benzoic acid, p-hydroxybenzoic acid, gallic acid, vanillic acid, syringic acid, o-coumaric acid, m-coumaric acid, p-coumaric acid, caffeic acid, ferulic acid, sinapinic acid, chlorogenic acid, and ellagic acid.

10. The topical composition of claim 1, further comprising at least one of a moisturizer, a sunscreen, an emollient, an emulsifier, a colorant, and a fragrance.

11. The topical composition of claim 1, further comprising an antioxidant.

12. The topical composition of claim 1, further comprising an additive, wherein the additive comprises at least one of argerline, ceramides, hyaluronic acid, niacinamide B3, red clover flower extract, retinol, azelaic acid, lactic acid, Matrixyl, and Nigerian hibiscus.

13. The topical composition of claim 1, wherein the polyphenol is at least one proanthocyanidin in a cranberry extract, and the phenolic acid is protocatechuic acid.

14. The topical composition of claim 13, wherein the topical composition comprises 10 mg/mL of the fructooligosaccharide, about 0.0001 mg/mL of the cranberry extract, and 0.6 mg/mL of the protocatechuic acid.

15. The topical composition of claim 13, wherein the cranberry extract comprises at least 15% by weight of the at least one proanthocyanidin.

16. The topical composition of claim 1, wherein the prebiotic is 10 mg/mL fructooligosaccharide, the polyphenol is at least 15% by weight of the proanthocyanidin in approximately 0.0001 mg/mL of a cranberry extract, and the phenolic acid is 0.6 mg/mL protocatechuic acid.

17. The topical composition of claim 1, wherein the prebiotic is 10 mg/mL of the fructooligosaccharide, the polyphenol is about 0.000015 mg/mL of the at least one proanthocyanidin, and the phenolic acid is 0.6 mg/mL of the protocatechuic acid.

18. The topical composition of claim 1, wherein the prebiotic is fructooligosaccharide in a range from 10 mg/mL to 25 mg/mL; the polyphenol is at least one proanthocyanidin in about 0.000015 mg/mL, and the phenolic acid is protocatechuic acid in a range from 0.3 mg/mL to 0.6 mg/mL.

19. The topical composition of claim 1, wherein the prebiotic is in a range from 10 mg/mL to 25 mg/mL, the polyphenol is about 0.000015 mg/mL, and the phenolic acid is in a range from 0.3 mg/mL to 0.6 mg/mL.

20. A topical delivery medium comprising the topical composition of claim 1, wherein the topical delivery medium is formulated to cause transdermal delivery of the topical composition to the skin.
